(19) United States
(12) Patent Application Publication
(10) Pub. No.: US 2021/0021787 A1
(43) Pub. Date: Jan. 21, 2021

(54) AUDIO-VIDEO CONFERENCING SYSTEM OF TELEMEDICINE

(71) Applicant: Thomas Lipp, Leipzig (DE)

(72) Inventor: Thomas Lipp, Leipzig (DE)

(21) Appl. No.: 16/962,840

(22) PCT Filed: Jan. 8, 2020
(86) PCT No.: PCT/DE2020/000002

§ 371 Date: Jul. 16, 2020

(30) Foreign Application Priority Data
Jan. 9, 2019 (DE) 10 2019 000 067.6

(51) Int. Cl. H04N 7/15; H04L 29/08; H04W 88/08; G16H 80/00; G16H 10/60; G16H 40/67; G16H 15/00; G16H 40/20; A61B 5/00; A61B 7/04; A61B 5/1455; A61B 1/227; A61B 1/31; A61B 5/0205; A61B 5/145

(52) U.S. Cl. CPC H04N 7/15; A61B 5/02438; H04W 88/08; G16H 80/00; G16H 10/60; G16H 40/67; G16H 15/00; G16H 40/20; A61B 5/7465; A61B 5/0022; A61B 7/04; A61B 5/14551; A61B 5/441; A61B 1/227; A61B 1/31; A61B 5/02055; A61B 5/14532; H04L 67/12

(57) ABSTRACT

The invention relates to an audio-video conference system for telemedicine, which in each case comprises a video conference end device at a base station which is located at the medical practice location, and an external station, which is located at a patient's location.

### CROSS REFERENCE TO RELATED APPLICATIONS

This application is a national phase of the International Patent Application PCT/DE2020/000002 filed on Jan. 8, 2020 that claims the priority from the German patent application 10219000067.6 dated Jan. 9, 2019.

### BACKGROUND OF THE INVENTION

The invention relates to an audio-video conference system for tele-medicine, which includes a video conference terminal device at a base station arranged at the medical practice location, and an external station arranged at the patient's location.

Currently there is a line of audio-visual conference systems for telemedicine in use, wherein the vital signs of a patient at the patient's location is measured through a nurse or the like and the data is subsequently transferred to a database at the clinic location. There, the main doctor calls up the measured vital signs from the database and assesses them.

Big cities and poorly served areas, mostly outlying and rural, as well as nursing homes and assisted living arrangements, are often times underserved by family doctors, especially specialist doctors, resulting in dissatisfaction, increased emergency situations, increased ambulance transport, and poorer and costlier medical treatment.

Currently, for example, nurses or medically trained personnel are being dispatched to patient's locations outside of the medical practice and using customary medical measuring devices to record patient data to subsequently send back to the medical practice in electronic form.

Subsequently, the nurse contacts the doctor from the patient's location and establishes communication between the doctor and the patient. The doctor has no means to actively influence or monitor the manner of the data collection. Furthermore, none of the patient data is in real time, creating a clear reduction to the quality of care the doctor can provide.

Besides, it is not possible to bring in further field expertise in real time, for example including a specialist doctor to improve the quality or efficiency of the diagnosis.

### SUMMARY OF THE INVENTION

The object of the invention is to create a technically simple, secure and robust audio-video conference system with a wireless transmission device of patient data, as well as providing measured patient data, which in particular enables a broadband connection for an audio-video connection between two or more locations, implementing a secure real-time audio-visual video conference in order to enable a prompt, high-quality medical diagnosis. Thereby it should be made possible to present the patient vital data to the doctor at the medical practice, and if necessary, to a specialist doctor at a further location, at the same time of the audio-visual video conference.

According to an aspect of the invention, an audio-video conference system is provided, comprising a conference end device at a base station, located at a medical practice location, and an external station, located at a patient's location, wherein at least one additional station is connectable to the base station, wherein the audio-video conference system has a transmission device for the wireless transmission of patient data in real time from the external station to the base station and/or an additional station, wherein the external station comprises at least one data interface for receiving of patient data, which are obtained through measurement using at least one medical measurement device on the patient, and the external station, which comprises at least one medical measurement device, is portable.

According to another aspect of the invention, a method for the implementation of an audio-video conference and the availability of patient measurement data at at least one base station in real time is provided, wherein at least one communication connection between the base station and the external station is established through an operator, the collection and transmission of patient data to the base station in real time is initiated by the operator, the operator working at the base station monitors the process steps and/or to prepare and collect measurements from the patient, and the operator examines and evaluates the transmitted patient data is conducted through.

### DETAILED DESCRIPTION OF THE INVENTION

The technical contribution is that the audio-video conference system features a transmission device 1 for the wireless transmission of patient data in real time from the external station 3 to the base station 2 and/or an additional station 4, wherein the external station 3 includes at least one data interface for the collection of patient data, which is determined by means of measurement using at least one medical measurement device 8 on the patient 9, and the external station 3 including at least one medical measurement device 8 is able to be carried.

Thereby it is made possible that the collected patient data is transmitted digitally to the external station in the usual manner and can be transmitted to the base station in the usual manner. These can regularly be encrypted in a known manner. The patient measurement data can therefore be made available to the family doctor and specialist doctor, as necessary, in a high-quality manner during the video conference.

For the purpose of the invention, the base station is at the location at which the treating doctor, namely the operator 5, is at during the time of the videoconference, specifically the medical practice of the doctor. Thereby the additional station can also be a mobile one, for example a smartphone or tablet.

For the purpose of the invention, the additional station is at the location of a specialist medical practice, where the treating specialist, namely operator 7, is at during the time of the videoconference. Thereby the additional station can also be a mobile one, for example a smartphone or tablet.

For the purpose of the invention the external station is at the location where the patient is, at which the to be treated patient and the operator 7 are at the time of the video conference.

With the invented transmission device it is made possible to transfer patient data from an external station 3 to a different location in a simpler manner, specifically to a base station 2, to facilitate recognizable data for the doctor in real time.

This technical solution make it possible for an easy, secure, and robust communication connection, for example through the use of a broad band connection for an AV-connection (audio-video) between at least one base station 2, an external station 3, and at least one additional station 4.

The additional station 4 is an external interdisciplinary tele-consultation point, which especially involves the following specialist fields:

Ear-nose-throat medicine,
Gastroenterology (for example colonoscopy),
Gynecology (outer),

### Dermatology,

### Cardiology,

### Orthopedics, Psychology, Neurology,

Urology (outer), and

### Surgery

### Psychiatry

### Psychotherapy

In connection with a telephone conference, involvement of further specialized medical expertise is possible, such as pharmacy or other service providers in the medical field.

Transmission devices for wireless transmission of patient data are provided, with at least one base station located at the medical practice location and at least one external station located at the patient's location, wherein the external station includes at least one data interface for the collection of patient data, which is obtained through measurement using at least one measurement device on a living subject. The data transmission ensues via all current relevant conventional media forms, particularly via mail and/or a livestream.

For the purpose there is someone on-site, namely at the external station, that is a non-medical assistant trained by us or an experienced nurse, who hosts consultation hours at specific times using a corresponding tablet (the patient comes to the video conference appointment), drives to house visits or contacts nursing homes. In our model the pure video conference appointment, namely the visual contact between patients and doctor established by the non-medical assistant, is supplemented by diagnosis of body surfaces and body cavities of a patient imageable by camera.

The solution can comprise a camera technology unit with a microprocessor, for example a PC, and a software solution for the transmission of pictures and films in real time, a telephone connection and various technical modules. Through that it can convey by teletransmission blood pressure, blood sugar, blood gas, ECG, and the inspection of the throat, the mouth, the ear, the nose area, as well as the inspection of skin and wounds, the outer genitals and the anus, gait, motion sequences, oxygen saturation, pulse, lung sounds, heart murmurs.

In the image data, patient information is regularly not sent. For these measurements it is necessary that the respective measurement device is connected to the subject, or that the respective measured quantities are measured, recorded, often also further processed and passed along in a more suitable, typical manner.

Whether the measurement requires contact with the patient or can be done remotely depends on the body properties, the body function or the vital sign parameters to be measured, as well as their corresponding measurement methods.

The doctors participating in teleconsulting, identified as operator 5 and 7, can converse about the patient over video conference or telephone.

Video appointments via video conference concerning patient contact can be made possible relatively problem-free technically. These serve primarily to monitor chronic illnesses, and in the case of acute illnesses, to decide if direct doctor-patient contact is actually necessary, if a hospital visit is in order, if on-site non-medical assistant, nurse, caregiver or telemedical family doctor can provide treatment, or if the patient needs to visit a specialist doctor.

This allows for moderation through application of adequate telemedicine by family doctors and specialist doctors exercising specialty medical expertise in real time through livestream audio-video conferences and direct diagnosis.

Telemedicine does not replace personal doctor-patient contact permanently or principally. The exception is in areas without access to medical care. In that case, the care can essentially be carried out exclusively and sufficiently in this manner of care, except for emergencies and serious illnesses.

Furthermore, a convenient telemedicine unit enables patients in nursing homes, assisted living facilities, and external bed-ridden patients to be treated by specialist medicine from a distance.

It is further enabled to conduct a telephone conference and switch between doctor and patients and/or the operator 7, namely a medically-trained individual, specifically a specialist doctor.

In the scope of the invention, the conference end device can be a typical PC, laptop, tablet, or mobile phone (smartphone), which provides conduction of an audio-video conference and measurement value display by means of a typical software.

The dependent claims 2 to 7 contain, without limitation, advantageous embodiments of the invention.

Preferably, at least one additional station 4 is connectable to the base station 2 in real time, wherein the operator 7 of the additional station 4 provides the patient data visually and/or audibly.

Specialist fields of the operator 7 should in particular be: surgeon, urologist, neurologist, psychiatrist, gynecologist, ENT, dermatologist, physiologist, orthopedist, psychologist, and/or cardiologist.

Thereby it is enabled to present visual, endoscopical, and dermatological findings to the connected specialist doctor via real-time video, and to collaboratively make the diagnosis and prescribe appropriate therapy.

The visual examination includes body surfaces and body cavities of a patient imageable by camera.

Therein it can relate to a specialist doctor-to-specialist doctor connection, family doctor-to-specialist doctor connection, specialist doctor-to-family doctor connection, or nursing home-to-doctor connection.

Furthermore, it is conceivable to establish connection between service provides (pharmacies, hearing device producers, psychotherapists, psychologists, ergotherapists) and specialists doctors.

Preferably, the external station 3 has a maximum weight of 10 kg, and is able to be carried by one person, specifically the operator 6.

Preferably, the external station 3 is technically simple, structurally small, and able to be easily transported by a female in order to minimize transport effort.

The manner of the selection of technical structural components of the external station 3 is limited accordingly.

Such technical structural components of the external station 3 are for example a laptop, a mobile phone, a mobile phone endoscopy attachment, a mobile phone dermatoscope attachment, and an endoscope. Internet access with at least ADSL 6000 transmission quality is necessary. The solution is in accordance with the principles of the current code of conduct and general legal requirements (such as data security).

Preferably, the external station 3 comprises at least one digital camera.

Preferably, the external station 3 comprises at least one ECG, particularly a smartphone-/smartwatch-ECG, a stethoscope, a dermatoscope, an endoscope, a digital camera and a pulse oximeter, which each includes a data interface for a wired and/or wireless transmission of measured vital signs over the data interface to an external station 3.

As a result of extensive tests, the existing selection of aforementioned electronic devices was made. Therewith, the external station should only feature devices that are statistically often needed for examination and generally can be carried to the patient's location by an operator 6, who is often female. Accordingly, the external station should have a maximum weight of 10 kg.

The aforementioned components of the external station could be stored in a typical transportation case, such as a backpack, suitcase, or the like.

A digital camera that may be used in the invention is a camera that utilizes a digital storage medium as recording medium as opposed to film; the picture is digitalized by means of an electronic image converter. Digital cameras are integrated into other devices. All modern mobile telephones and smartphones include a built-in digital camera.

Preferably, a broad band connection exists for at least one audio-video connection between at least a base station 2 and an additional station 4. The scope of the invention includes at least one audio-video connection, at least such as is currently typical and known in Germany.

Preferably, the base station 2, the external station 3 and the additional station 4 respectively include at least a sending, receiving, display, and/or playback device for visual and/or audio signals and/or patient data.

The object of the invention is solved through a process with the features according to claim 8.

The technical contribution is that the process for the provision of patient data of living subjects to at least one base station 2 in real time, wherein a least one communication connection, specifically an A/V-connection, is established between the base station 2 and the external station 3 by an operator 6, the operator 6 initiates the transmission of patient data to the base station 2 in real time, the operator 5 working at the base station 2 is enabled to monitor the process steps and/or to prepare and collect measurements from the patient, and through the operator 5 an examination and/or an evaluation of the transmitted patient data (and initiating further activities) ensues.

It is enabled that during the general audio-video conference, visual contact exists between doctor (also particularly the family doctor/primarily addressed doctor) and patient.

The dependent claims 9 and 11 contain advantageous embodiment of the invention without imposing limitations.

Preferably, the operator 6 of the external station 3 is a nurse, a patient 9, a caregiver, a relative of patient 9, or a different technically trained person.

The object of the invention is solved by an inventive transmission device with the features according to claim 12.

A possible application could be the obtainment of a second opinion in the presence of unclear or complicated findings.

A personal introduction of the patient is independent thereof, and possible at any time.

In order to secure the immediate contact of a video conference, each field should have multiple specialist doctors participating and selectable. It is also possible to include a specialist doctor retrospectively.

### EXAMPLE 1

### Process Description of a Tele-Consultation During an ENT Illness

A nurse, namely an operator 6, drives to a patient at the patient's location, which for example could be a nursing home or the patient's living quarters, by means of a car, moped, or bicycle.

The nurse establishes livestream contact to the family doctor or an operator 5, who is at the base station 2 located at the medical practice. Therewith, livestream contact is established between the patient, the nurse, and the doctor. Therewith, visual contact between doctor and patient can be established, whereby for example an integrated digital camera of an iPad or iPhone positioned on a stand is used in typical manner.

Subsequently, the patient is examined under the instruction of the active doctor.

The findings are assessed in real time, for example by ECG etc. is available to the doctor in real time.

The doctor can, if appropriate, control instruments—under remote visual contact—by relaying corresponding instructions to the nurse, for example an endoscope/camera, by means of the nurse, virtually guiding their hand, looking into the throat or ear, i.e. the doctor does not merely view transmitted images, but rather the findings directly, for example skin spots, and controls the tele-technology by means of the nurse, who effectively acts as an extended arm for the doctor, who essentially serves as a direct assistant.

The doctor is also present “on-site”, speaking as needed, and optionally switching the livestream to other doctors, conferring directly with specialists, essentially creating a 3-party video conference: doctor-patient/nurse-specialist.

This results in an availability of patient data to at least one base station 2 in real time, wherein at least one communication connection is established, specifically an A/V-connection, between the base station 2 and the external station 3 through an operator 6.

The operator 5 working at the base station 2 is enabled to monitor the process steps and/or to prepare and collect measurements from the patient. Therewith, the operator 5 is enabled by the family doctor to carry out an examination and assessment of the transmitted patient data in real time and, if necessary, consult with a specialist doctor in real time.

The on-duty specialist doctors can, for example, be marked available in a computer-based database so that they can be added to the connection in real time.

In additional, a communications connection to an external specialist doctor is established, wherein at least one additional station 4 is connected to the base station 2 in real time. The operator 7, i.e. the specialist-doctor, is provided at least a part of the patient data visually and/or audibly via the additional station 4, which is connected to the base station 2.

Accordingly, a “direct” consulting room is established, wherein the patient sits “across from” the doctor, and the nurse is physically present to directly carry out the doctor's instructions. Furthermore, the specialist doctor comes into the room virtually and a consultation is conducted in the presence of the patient.
1) Data transfer from vital signs to the doctor: temperature, blood pressure, blood sugar, pulsoximeter, weight (scale), spirometer, ECG, etc.
2) Transmission of images: dermatoscopy/camera
3) Transmission of (live) videos: endoscopy/camera

### Example A

Technical Requirements:

Broad band connection for an AV-connection (audio-video) between two or more medical practice locations.

### EXAMPLE B

Technical Requirements:
Clinic PC, Headset
Access to at least 6000 ADSL access
Data safe (server)
Endoscopic unit: Telepack
Endoscope (ENT)
Dermascope (derma)
Rectoscope (uro)
A second connectable camera (for example for tele-endoscopy)

### EXAMPLE 2

### Process Description of a Tele-Consultation During an ENT Illness

A patient goes to a general/family doctor due to ENT complaints. The doctor meets with the specialist doctor via a tele-consultation connection, examines the patients with an endoscope with the help of the specialist doctor and discusses the diagnosis with the ENT doctor. The digital camera of the mobile telephone (smartphone) or tablet is placed on a stand and fixated on the patient, so that the doctor can see the patient. The nurse takes the measurement of the patient in typical manner by means of one of the measurement devices of the external station. The collected patient measurements are separated i.e. sent in part via the digital camera of a mobile telephone (smartphone) or tablet, to the family doctor and/or specialist doctor, in real time.

The general doctor prompts any further necessary examinations. The patient then goes to the ENT doctor for therapy, if still necessary, with all the findings from the conducted examination. This presents a multitude of advantages for the patient: he already receives specialist help during his first doctor visit, he experiences reduced travel and wait times at the general and specialist doctors, as well as potential for a streamlined treatment process.

### EXAMPLE 3

### Process Description of a Tele-Consultation During an ENT Illness

An operator 6, for example a patient 9, establishes contact via livestream with their family doctor, namely an operator 5, who is at the base station 2 at the medical clinic. Thereby, contact is established via livestream between the patient 9 and the doctor.

Thereby, visual contact can be established between doctor and patient, wherein, for example, a digital camera integrated in an iPad or iPhone is positioned on a stand in a usual manner.

Alternatively, a standard computer with a digital camera can act as the external station 3 in typical manner.

Subsequently, the conduction of a patient examination ensues under the instruction of the active doctor.

The examination's findings follow in real time and are made available to the doctor in real time.

The doctor can, if appropriate, control instruments, in particular the digital camera, by instructing the patient under visual supervision, accordingly the doctor virtually guides the hand of the patient 9, looks into the throat or ear i.e., the doctor looks at findings, for example a skin spot, directly rather than merely viewing transmitted images, and controls the tele-technology, which operates as an extended arm of the doctor, basically rendering the patient 9 a direct assistant to the doctor.

The doctor is also present “on-site”, speaking as needed, and optionally switching the livestream in addition to other doctors, conferring directly with specialists, essentially creating a 3-party video conference: doctor-patient/nurse-specialist.

Thereby the patient data is made available in real time to at least this base station 2, wherein an operator 6 establishes at least one communication connection, specifically an A/V-connection, between the base station 2 and the external station 3.

The operator 5 working at the base station 2 is enabled to monitor the process steps and/or to prepare and collect measurements from the patient. Thus, the family doctor, namely operator 5, can perform an examination and assessment of patient data in real time, and if necessary, confer with a specialist doctor in real time.

Thereto a communication connection to an external specialist doctor can be established, whereby therein at least one additional station 4 is connected to the base station in real time. The operator 7 i.e. the specialist doctor, has at least a portion of the patient data transmitted visually and/or audibly through the additional station 4, which is connected to the base station 2.

Therewith a “direct” consultation room is established, where the patient 9 sits “across from” the doctor. Further, the specialist doctor comes into the room virtually and a consultation is conducted in the presence of the patient.

What is claimed is:

1. An audio-video conference system, comprising a conference end device at a base station, located at a medical practice location, and an external station, located at a patient's location, wherein at least one additional station is connectable to the base station, wherein the audio-video conference system has a transmission device for the wireless transmission of patient data in real time from the external station to the base station and/or an additional station, wherein the external station comprises at least one data interface for receiving of patient data, which are obtained through measurement using at least one medical measurement device on the patient, and the external station, which comprises at least one medical measurement device, is portable.

2. The audio-video conference system of claim 1, wherein at least one additional station is connectable to the base station in real time, wherein the operator of the additional station presents the patient data visually and/or audibly.

3. The audio-video conference system of claim 1, wherein the external station has a maximum weight of 10 kg, and is portable by one person, namely the operator.

4. The audio-video conference system of claim 1, wherein the base station, the external station, and the additional station respectively have at least one send, receive, display and/or playback feature for visual and/or audible signals and/or measurement data.

5. The audio-video conference system of claim 1, wherein the external station comprises at least one of: an ECG, in particular a smartphone/smartwatch-ECG, a stethoscope, a dermatoscope, an endoscope, a digital camera and a pulsoximeter, which respectively include a data interface for wired and/or wireless transmission of measured vital signs via the data interface to the external station.

6. The audio-video conference system of claim 1, wherein the external station comprises at least one digital camera and one pulsoximeter, which respectively include a data interface for wired and/or wireless transmission of measured vital signs via the data interface to the external station.

7. The audio-video conference system of claim 1, wherein the external station includes at least one digital camera and one data interface for wired and/or wireless transmission of measured vital signs via the data interface to the external station.

8. A method for the implementation of an audio-video conference and the availability of patient measurement data at at least one base station in real time, wherein at least one communication connection between the base station and the external station is established through an operator, the collection and transmission of patient data to the base station in real time is initiated by the operator, the operator working at the base station monitors the process steps and/or to prepare and collect measurements from the patient, and the operator examines and evaluates the transmitted patient data is conducted through.

9. The method of claim 8, wherein for the examination and evaluation of the patient data, a connection from the base station to at least one additional station is established in real time and the operator of the additional station evaluates the patient data in real time.

10. The method of claim 8, wherein the evaluation of the findings is carried out in real time by the operator and/or the operator.

11. The method of claim 8, wherein the operator of the external station is a nurse, a patient, a caregiver, a relative of the patient and/or another technically instructed person.

12. Use of the audio-video conference systems comprising a conference end device at a base station, located at a medical practice location, and an external station, located at a patient's location, wherein at least one additional station is connectable to the base station, wherein the audio-video conference system has a transmission device for the wireless transmission of patient data in real time from the external station to the base station and/or an additional station, wherein the external station comprises at least one data interface for receiving of patient data, which are obtained through measurement using at least one medical measurement device on the patient, and the external station, which comprises at least one medical measurement device, is portable, wherein said use is for audio-video conferences and measured data transmission for tele-medicine in real time.
